(19) United States
(12) Patent Application Publication
(10) Pub. No.: US 2011/0287059 A1
(43) Pub. Date: Nov. 24, 2011

(54) HONEY ANALYSIS

(75) Inventors: Jonathan Counsell Stephens, Hamilton (NZ); Ralf-Christian Schlothauer, Tauranga (NZ)

(73) Assignee: COMVITA NEW ZEALAND LIMITED, Paengaroa, Te Puke (NZ)

(21) Appl. No.: 13/141,287

(22) PCT Filed: Dec. 23, 2009
(86) PCT No.: PCT/NZ2009/000301

§ 371 Date: Aug. 9, 2011

(30) Foreign Application Priority Data
Dec. 24, 2008 (NZ) NZ581615
Nov. 2, 2009 (NZ) NZ572474

(51) Int. Cl. A61K 35/64; G01N 33/00; A61P 31/00; A61P 29/00; A61P 37/04

(52) U.S. Cl. 424/278.1; 424/537; 73/61.43

(57) ABSTRACT

A number of methods of analysing honey are described to determine the age of the honey, the presence of fortification with additives including methylglyoxal (MGO), the region from which the honey is derived, the plant species from which the honey is derived and whether the honey has been heated during processing. The various characteristics are determined based on the phenolic concentrations in the honey which have been found to provide very clear markers for the above characteristics. The methods of analysis described have many applications, particularly around quality assurance and ensuring that honeys are true to labeled specifications.

### TECHNICAL FIELD

The application relates to honey analysis. More specifically, the application relates to methods of analysing honey to measure the medical and nutritional potency of the honey.

### BACKGROUND ART

Over the last 40-50 years bacteria have become increasingly resistant to commonly used antibiotics. As a result many infections previously readily cured by antibiotics are now difficult or impossible to treat (Finch, R. G. (1998) Antibiotic resistance. Journal of Antimicrobial Chemotherapy 42, 12.5-128). Given this, empirical screening of chemical entities for antimicrobial activity represents an important strategy for the development of novel drugs. Natural products in particular have been a rich source of antimicrobial agents, that in general are associated with low levels of toxicity, and in many cases have a fairly broad spectrum of activity (Silver, L. and Bostian, K. (1990) Screening of natural-products for antimicrobial agents, European Journal of Clinical Microbiology & Infectious Diseases 9, 455-461). A natural product that has received significant attention due to its anti-bacterial action is honey. Although honey has been used for the treatment of respiratory infections and for the healing of wounds since ancient times (Moellering R C. (1995). Past present and future antimicrobial agents. American J Medicine, 1995; Supp 6A 11S-18S; Jones H R. Honey and healing through the ages. In Honey and Healing. ed Munn P A and Jones H R. 2001; 1-4. Cardiff, IBRA) it was not until the late 20th century, as a result of the increasing resistance of micro-organisms to antibiotics that research studies began to document the anti-bacterial activity of honey against a number of pathogens (Allen K L. Molan P C. Reid G M. (1991) A survey of the antibacterial activity of some New Zealand honeys. Journal of Pharmacy & Pharmacology. 43(12):817-22; Willix D J. Molan P C. Harfoot C G. (1992) A comparison of the sensitivity of wound-infecting species of bacteria to the antibacterial activity of manuka honey and other honey. Journal of Applied Bacteriology. 73(5)388-94). While the majority of honeys have been shown to have anti-bacterial activity, manuka honey, a honey produced by bees from the flowers of the manuka bush (Leptospermum scoparium) have been shown to possess the highest levels of anti-bacterial activity (Molan P C. The antibacterial activity of honey. 2. (1992). Variation in the potency of the antibacterial activity. Bee World 73: 59-76) and to be active against a range of pathogens including Staphylococcus aureus, coagulase-negative Staphylococci, Enterococci and Pseudomonas aeruginosa (Cooper R A. Molan P C. Harding K G. (1999). Antibacterial activity of honey against strains of Staphylococcus aureus from infected wounds. Journal of the Royal Society of Medicine, 92(6):283-5; Cooper R A, Halos E, Molan P C, (2002). The efficacy of honey in inhibiting strains of Pseudomonas aeruginosa from infected burns. J Burn Care Rehabil 23: 366-70; Cooper R A, Molan P C, Harding K G. (2002). Honey and gram positive cocci of clinical significance in wounds, J Appl Microbiol; 93: 857-63; V. M. French, R. A. Cooper and P. C. Molan, (2005). The antibacterial activity of honey against coagulase-negative staphylococci Journal of Antimicrobial Chemotherapy 56, 228-231). Indeed today manuka honey is a well accepted and established clinical treatment for infection associated with wounds and burns, where it has been shown to have both anti-infective and wound healing properties (Cooper 1999; Molan P C. Potential of honey AM J Clin Dermatol 2001; 243-19; A T Ali, M N Chowdhury, M S al Humayyd. (1991) Inhibitory effect of atural honey on Helicobacter pylori. Trop Gastroenterol).

In addition to its use for the treatment of wounds it has also been shown that manuka honey has antibacterial activity against the gastric pathogen H. pylori, the causative agent of gastritis and the major predisposing factor for peptic ulcer disease, gastric cancer and B-cell MALT lymphoma (N Al Somal K E Coley, PC Molan and B Hancock. (1994). Susceptibility of helicobacter pylori to the antibacterial activity of manuka honey. Journal of the Royal Society of medicine 1994; 87; 9-12; Soto Ms. Reddy S G. (1999) Graham D Y. Osmotic effect of honey on growth and viability of Helicobacter pylori. Digestive Diseases & Sciences. 44(3):462-4; Osato M S. Reddy S G. (1999) Graham D Y. Osmotic effect of honey on growth and viability of Helicobacter pylori. Digestive Diseases & Sciences. 44(3):462-4). Indeed a number of in vitro studies have shown that concentrations of manuka honey as low as 5-10% (v/v) can inhibit the growth of H. pylori (Somal 1994, Soto 1999, Osato 1999). This finding is of particular interest given that over recent years resistance to currently available antimicrobial agents against H. pylori has increased dramatically leading to an increasing number of treatment failures (L. Fischbach; E. L. Evans. (2007) Meta-analysis: The Effect of Antibiotic Resistance Status on the Efficacy of Triple and Quadruple First-line Therapies for Helicobacter pylori Aliment Pharmacol Ther.; 26(3):343-357). Indeed, in some populations, the level of resistance to clarithromycin, one of the major antibiotics used in the treatment of H. pylori, has been reported to be as high as 30-40% in some countries and is commonly associated with treatment failure (Josette Raymond, Christophe Burucoa Olivier Pietrini Michel Bergeret Anne Decoster Abdul Wann, Christophe Dupont and Nicolas Kalach (2007) Clarithromycin Resistance in Helicobacter pylori Strains Isolated from French Children: Prevalence of the Different Mutations and Coexistence of Clones Harboring Two Different Mutations in the Same Biopsy helicobacter Volume 12 Issue 2 Page 157-163). Resistance to metronidazole, a second antibiotic commonly used in the treatment of H. pylori infection has also been reported to be high (30%-40% in US and Europe and >80% some countries of the developing world), although in some cases in vitro resistance does not translate into eradication failure (Raymond 2007, Elvira Marvic, Silvia Wittmann, Gerold Barth and Thomas Henlel (2008) Identification and quantification of methylglyoxal as the dominant antibacterial constituent of Manuka (Leptospermum scoparium) honeys from New Zealand Mol. Nutr. Food Res. 2008, 52, 000-000). Given this environment, alternative treatment approaches are of interest.

While the antimicrobial activity of honey has been reported to include osmolarity, acidity, hydrogen peroxide and plant-derived components, more recent studies have shown that osmolarity, acidity and hydrogen peroxide activity cannot account for all of the honey activity, and that enhanced activity may be due to phytochemicals found in particular honeys, including manuka honey (Molan 1992). For example Cooper et al. (Cooper 1999) in a study of the antibacterial activity of honey against Staphylococcus aureus isolated from infected wounds showed that the antibacterial action of honey in infected wounds does not depend wholly on its high osmolarity, and suggested that the action of manuka honey stemmed partly from a phytochemical component (Cooper 1999).

Until recently the identity of these phytochemicals in manuka honey remained unclear, however in 2008 a study by Marvic et al reported that the pronounced antibacterial activity found in manuka honey directly originated from a chemical compound, methylglyoxal (MGO). In this study six samples of manuka honey were shown to contain over 70 times higher levels of methylglyoxal (up to 700 mg/kg) than that found in regular honeys (up to 10 mg/kg) (White, J. W., Schepartz, A. I. and Subers, M. H. (1963) Identification of Inhibine, Antibacterial Factor in Honey, as Hydrogen Peroxide and Its Origin in a Honey Glucose-Oxidase System. Biochimica Et Biophysica Acta 73, 57).

### Floral Markers

As noted above, phytochemicals are thought to have an Important role in relation to activity. Honeys have been known for some time to include a variety of phenolic compounds, flavonoids and abscisic acid. A selection of prior art on this point includes the following documents:

Ferreres et al 1996 (Ferreres et at Natural occurrence of abscisic acid in heather honey and floral nectar. J. Agric. Food Chem, 1996 44, 2053-2056) describes tests done on heather honey to find two non-flavonoid components as the main constituents being two isomers of abscisic acid. The corresponding flower nectar from which the honey is derived was also found to contain both isomers as the main constituents. This document notes that the abscisic acid isomers were not detected in other monofloral honey samples so Ferreres suggests that abscisic acid may be used as a marker for heather honey.

Gheldof et al June 2002 (Nele Gheldof, Xiao-Hong Wang and NickiJ Engeseth (2002) Identification and Quantification of Antioxidant Components of Honeys from Various Floral Sources, J. Agric. Food Chem 2002 so, 5870-5877) describes tests completed on honeys for antioxidant capacity and phenolic content. Antioxidant content was found to be proportional to phenolic content and darker honeys such as buckwheat were found to have high antioxidant capacities. This application suggests that the phenolic content of honey may be used as an indicator of honey origin.

Gheldof et al 2002a (Nele Gheldof and Nicki J Engeseth (2002) Antioxidant Capacity of Honeys from Various Floral Sources Based on the Determination of Oxygen Radical Absorbance Capacity and Inhibition of in Vitro Lipoprotein Oxidation in Human Serum Samples J. Agric. Food Chem 2002 50, 3050-3055) describes further experimentation completed from the earlier article. The aim in this article was to characterise the phenolics and other antioxidants in the honeys tested. In this article the authors found that honeys have similar types of antioxidants but different amounts of phenolic compounds. The author concluded that the phenolics were significant to antioxidant capacity but not solely responsible. Examples of antioxidant materials noted included proteins, gluconic acid, ascorbic acid, hydroxymethylfurfuraldehyde and enzymes such as glucose oxidase, catalase and peroxidase. Barberan et al 1993 (Francisco A. Tomas-Barberan, Frederico Ferreres, Cristina Garcia-Viguera, and Francisco Tomas-Lorente (1993) Flavonoids in honey of different geographical origin. Z Lebensm Unters Forsch 196:38-44) describes analysis of flavonoids in honey. The authors of this article found that flavonoids were incorporated into honey from propolis, nectar or pollen and that honeys from the northern hemisphere tended to show higher degrees of propolis based flavonoids while equatorial and Australian based honeys were largely devoid of propolis based flavonoids. South American and New Zealand honeys contained flavonoids associated with propolis.

Yao et al 2003 (Lihu Yao, Nivedita Datta, Francisco A. Tomas-Barberan, Federico Ferreres, Isabel Martos, Riantong Singanusong (2003) Flavonoids, phenolic acids and abscisic acid in Australian and New Zealand Leptospermum honeys. Food Chemistry 81 (2003)159-168) describes the use of measuring flavonoid, phenolic acid and abscisic acid content in Australian and New Zealand honeys as a method of authenticating honey floral origins. The authors found that Australian jelly bush honey included myricetin, luteolin and tricetin as the main flavonoids. Phenolics were found to be primarily gallic and coumaric acids along with abscisic acid. By contrast New Zealand manuka honey contained quercetin, isorhamnetin, chrysin, luteolin and an unknown flavanin. The main phenolic compound was found to be gallic acid. In addition, almost three times the amount of abscisic acid was found in New Zealand manuka honey as Australian jelly bush honey.

Barberan et al 2001 (Francisco A Tomas-Barberan, Isabel Martos, Federico Ferreres, Branka S Radovic and Elke Anklam (2001) HPLC flavonoid profiles as markers for the botanical origin of European unifloral honeys. J Sci Food Agric 81:485-496) describes how the phenolic profiles of 52 honeys from Europe were analysed. The different honeys were found to have different markers with different characteristics and UV spectra. Different markers however were found to be present in several honeys rather than being specific to one species. For example, abscisic acid was found in heather honey, rapeseed, lime tree and acacia honeys.

As should be appreciated from the above, a variety of experiments have been undertaken to determine characterising compounds in honeys. Knowledge exists that honey contains antioxidant activity and that this may be attributable to compounds such as flavonoids, phenolic acids and abscisic acid. What should also be apparent from the above is that different studies have found that these compounds are present in a variety of honeys and that the amount present and the types of compound present may be a misleading measure of the honey origin due to their variation and lack of correlation between plant and honey. For example, abscisic acid is found in a variety of different honeys from different plant species but the quantities vary substantially even between samples from the same source.

The authors of the above documents do not consider whether honey age has any influence on the composition of the various compounds analysed.

### Methoxylation

Most dietary polyphenols have very poor bioavailability due faster metabolic breakdown of hydroxyl groups as opposed to methoxyl groups. Methoxylated phenolics are highly resistant to human hepatic metabolism (Wen, X., Walle, T. (2006a) Methylation protects dietary flavonoids from rapid hepatic metabolism. Xenobiotica 36; 387-397) and also have much improved intestinal transcellular absorption (Wen, X., Walle, T. (2006b) Methylated flavonoids have greatly improved intestinal absorption and metabolic stability. Drug Metab. Dispos. 34: 1786-1792). The methylated flavones show an approximately 5- to 8-fold higher apparent permeability into cells which makes them much more bio-available. The higher hepatic metabolic stability and intestinal absorption of the methylated polyphenols make them more favourable than the unmethylated polyphenols for use as potential cancer chemo-preventive agents. The determination of metabolic stability of four methylated and their corresponding unmethylated flavones with various chemical structures all of the tested methylated flavones, showed much higher metabolic stability than their corresponding unmethylated analogues.

It should be appreciated from the above that it would be useful to have a means for adjusting the level of medical and/or nutritional potency of honey. Since plants from which honeys are derived contain key compounds with medical and nutritional potency, it should further be appreciated that methods of manipulating plants to enhance key compound levels would be useful. It is an object of the instant application to address the foregoing problems or at least to provide the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the embodiments described herein will become apparent from the ensuing description that is given by way of example only.

### BRIEF DESCRIPTION OF THE DRAWINGS

Further aspects will become apparent from the following description which is given by way of example only and with reference to the accompanying drawings in which;

FIG. 1 shows a graph illustrating the phenolic profile of monofloral manuka, kanuka, and other honeys harvested in New Zealand and aged naturally for up to ten years;

FIG. 2 shows a graph illustrating the correlation between the sum of the principal phenolic components and methylglyoxal in monofloral manuka honey harvested in New Zealand and naturally aged;

FIG. 3 shows a graph illustrating the presence of selected phenolic compounds in plant nectar for four different plants used in honey production;

FIG. 4 shows a graph illustrating the concentration of methylglyoxal in naturally aged manuka honey and two manuka honey samples that have been artificially heated to release methylglyoxal;

FIG. 5 shows a graph illustrating the concentration of the principal phenolic components and methylglyoxal in naturally aged manuka honey, and two manuka honey samples that have been artificially heated to release methylglyoxal;

FIG. 6 shows a graph illustrating the correlations between the sum of principal phenolic components in manuka and kanuka honey and honey age;

FIG. 7 shows a graph illustrating the impact of heat on phenolic compounds and MGO using paired samples in manuka honey, 25% clover honey and 25% rewarewa honey blends with the same manuka honey. % concentration change represents increase of described component after 50 days treatment relative to initial concentration;

FIG. 8 shows a graph compairing paired samples illustrating the effect of long-term storage at room temperature on the concentration of phenolic compounds and MGO in manuka honey, and 25% clover and 25% rewarewa blends of the same manuka honey. % concentration change represents increase of described component after so and Zoo days of storage; and,

FIG. 9 shows a graph compairing paired samples illustrating the effect of acidification and storage at room temperature on the concentration of phenolic compounds and MGO in manuka honey. % concentration change represents increase of described component after 50 and 200 days of storage.

### DETAILED DESCRIPTION

The instant application broadly relates to maintaining and/or maximising the medical and nutritional potency of honey by use of the finding that phenolic compounds in honey are a key driver in honey potency. Since the levels of phenolic compounds can be analysed and adjusted during honey manufacture, methods to produce greater numbers of phenolic compounds and therefore increased medical and nutritional potency are of interest. In addition, knowing the above properties allows for further quality control in honey manufacture.

Finding the above synergies was surprising as this goes against recent publications which suggest that methylglyoxal (MGO) is the primary compound and those which have found abscisic acid to be a major factor.

A further finding by the inventors was the fact that the free phenolic compounds concentrations change over time in the honey and in response to other factors such as heat and dilution. This change in concentration over time was unexpected and is thought to be the main reason why earlier trials looking at phenolic compounds were unsuccessful or gave mixed or inconsistent results.

Also contrary to the art was the inventors finding that in fact phenolic compounds in plant nectar (as opposed to pollen or other measures) was highly correlated to the levels found in honey sourced from the same plants. As noted above, the art finds no correlation between plant nectar phenolic compound levels and that observed in honey and therefore concludes that phenolics are not a useful measure. In contrast, when age is taken into consideration, phenolic compounds are highly correlated between plant nectar and honey. This finding has meant that it is possible for the inventors to measure a wide range of factors in the honey well beyond that speculated in the art of only origin.

The exact mechanism behind why the free phenolic levels vary in honey overtime is not certain however the inventor understands that these phenolic entities are initially carried by a tannin molecule(s) present in the nectar, and as the honey ages naturally the phenolic molecules are released due to degradation of the tannin body and the matrix associated with a large organic molecule with both hydrophobic and hydrophilic binding sites. The best researched comparison for such aging is the development of flavour and aroma in red wines due to the release of phenolic groups from tannin bodies.

The same result of an increase in MGO concentration overtime for honeys that include MGO e.g. manuka honey, was also measured by the inventors although other processing steps could be used to adjust MGO concentration and not adjust phenolic concentration hence a different mechanism of release appears to occur for MGO. Prior art suggests that this may be due to conversion of DHA into MGO as a natural reaction process within the honey and that this reaction is sensitive to age like phenolics as well as other influences e.g. heat and acidification.

The improved healing effects are in part thought to be due to these compounds offering or influencing multiple stages of healing. The different stages are an antimicrobial phase, an immune stimulation phase and an anti-inflammatory phase. All of these aspects are understood to contribute to potency of honey in medical and nutritional applications.

Methods of determining and manipulating characteristics associated with honey are now described including:
(a) the age of a honey;
(b) whether the honey has been fortified with MGO;
(c) whether the honey has been heated;
(d) whether honey has been acidified;
(e) which region a honey has been sourced from;
(f) which plant species the honey has been produced from.

For the purposes of this specification the term ‘phenolic compounds’ and grammatical variations thereof refers to phenolic acids, phenolic salts, phenolic esters and related polyphenolic compounds. The term ‘free’ in the context of phenolics refers to phenolic compounds being in a readily detectable form.

The term ‘complexed’ in the context of phenolics refers to phenolic compounds being carried in a tannin molecule or otherwise not detectable, for example as a result of in vivo phenolic self condensation or precipitation reactions occurring as a result of honey bees dehydrating nectar.

### Age

According to a first embodiment there is provided a method of determining the age of a honey sample by measuring the concentration of phenolic compounds in the honey and comparing this concentration to a honey with a known age.

As noted above, a key finding of the inventors is that the phenolic levels in honey change overtime. This finding means that it is possible to take an unknown honey, measure the level of phenolic compounds in the honey and by correlation, predict the age of the unknown honey sample. Based on the inventors work, this method exhibits 95% confidence interval accuracy.

In the above method, the phenolic compounds measured are free phenolic compounds.

An advantage of knowing the age of a honey sample is that the inventors have found that phenolic levels correspond to medical and nutritional potency hence knowing honey age allows for blending operations that tailor medical and nutritional efficacy.

### MGO/Heat/Acidification

According to a second embodiment there is provided a method of determining whether a honey has been fortified with MGO by:
(a) knowing the approximate age of the honey and measuring the concentration of phenolic compounds and/or MGO in the honey; and,
(b) comparing the measured concentration of phenolic compounds and/or MGO against a control honey with a known age.

In a further variation, both phenolic concentration and MGO concentration if present are both measured in the unknown honey and compared against a known control honey.

In the above embodiment, MGO fortified honey has a high MGO concentration and comparatively low phenolic concentration in proportion to an unheated honey.

According to a third embodiment there is provided a method of determining whether a honey has been heated by:
(a) knowing the approximate age of the honey and measuring the concentration of phenolic compounds and/or MGO in the honey; and
(b) comparing the measured concentration of phenolic compounds and/or MGO against a control honey with a known age.

In a further variation, both phenolic and MGO concentration are measured in the unknown honey and compared against a known control honey.

In the above embodiment, heated honey has a high MGO concentration and comparatively low phenolic concentration in proportion to an unheated honey.

According to a fourth embodiment there is provided a method of determining whether a honey has been acidified by:
(a) knowing the approximate age of the honey and measuring the concentration of phenolic compounds and/or MGO in the honey; and,
(b) comparing the measured concentration of phenolic compounds and/or MGO against a control honey with a known age.

In a further variation, both phenolic and MGO concentration are measured in the unknown honey and compared against a known control honey.

In the above embodiment, acidified honey has a high MGO concentration and comparatively low phenolic concentration in proportion to an unheated honey. This result is particularly pronounced over time.

As noted above, one example of an additive of concern is methylglyoxal (MGO) which has been the subject of recent reports attributing MGO to UMF activity and therefore giving some motivation for producers to fortify natural honey with MGO in order to increase the honey value.

Another example as noted above is that of heat during processing. Heating honey after collection can artificially increase the MGO content. Whilst the exact mechanism for this is not certain, it is understood by the inventors that heat releases MGO that may be bound in the honey sugars. Although an increased MGO level may be advantageous, heat can result in unwanted reactions occurring with honey and in particular the production of hydroxymethylfurfuraldehyde (HMF) compounds. Honey that has levels of HMF in excess of 40 ppm HMF can be downgraded as bakers honey or denied market access as HMF may be linked to harmful effects. Also of concern with heating is that fragile phenolic compounds understood by the inventors to be therapeutic may be deactivated or broken down by heat hence heat can be undesirable, particularly if this is not controlled.

At present there are no known methods to confirm post processing if MGO has been added to honey.

With heat in processing, HMF compounds can be detected where gross heating has occurred but more limited heating is harder to detect.

Embodiments of the instant application provide the opportunity to test for the above compounds.

In selected embodiments, it is possible to also quantitatively determine how much MGO has been added to a honey by the methods described above.

Similarly, in selected embodiments, it is possible to also quantitatively determine how much heat a honey has been subjected to by use of the methods described above.

It should be appreciated from the above description that there are described methods of identifying MGO fortified or heat treated honeys. The advantages which should be apparent to those skilled in the art include the ability to test for adulteration or manipulation of natural based honeys and therefore act as a quality standard.

An alternative advantage of the present invention is the ability to selectively adjust honey characteristics in a controlled and measurable way.

For example, it may be desirable to deliberately produce a high MGO content honey for fortification as with synthetic MGO but avoid the danger of having to much MGO present and therefore creating the risk of side effects or even of toxicity of MGO. Embodiments of the method allows the user to predict how much MGO may be added to achieve a desired anti-bacterial effect from the honey, especially against gram positive bacteria. It is understood that phenolic compounds tend to mitigate any potential free radical producing or gluthathione depletion effects from MGO. It is therefore important to dose MGO at a rate so as not to overwhelm the radical quenching and glutathione repletion effect that the phenolic compounds are understood to contribute.

In an alternative example, heat may be deliberately applied to a honey at a predetermined temperature in order to increase MGO content. Embodiments of the method described herein could be used to prevent the production of unwanted by-products from heat such as HMF compounds. This heating step may also be completed in order to increase the free phenolic content of the honey as heat is understood to be a mechanism to un-complex phenolics.

Finally it should be noted be noted that not all honeys contain MGO naturally. The above methods in respect of MGO are therefore best suited to honeys with MGO present naturally including but not limited to Leptospermum species.

### Honey Region

According to a fourth embodiment there is provided a method of determining the regional origin of a honey sample by knowing the approximate age of the honey and measuring the concentration of phenolic compounds in the honey and comparing the results to a control sample or samples.

According to a fifth embodiment there is provided a method of determining whether a honey sample is a blend of honeys from different regions by knowing the approximate age of the honey and measuring the concentration of phenolic compounds in the honey and comparing the results to a control sample or samples.

As noted above, phenolic compounds in honey may vary dependent on the region in which the honey is collected.

In one embodiment, honey derived from Leptospermum scoparium var. incanum and Leptospermum scoparium var. linifolium grown in parts of New Zealand can be distinguished from honey sourced from Leptospermum scoparium var. myrtifolium and Leptospermum scoparium var. ‘triketone’ on the basis on a comparison of methoxylated benzoic acids. Leptospermum scoparium var. incanum and Leptospermum scoparium var. linifolium derived, honeys are characterized by having significantly higher methoxylated benzoic acid levels than honey derived from the varieties Leptospermum scoparium var. myrtifolium and Leptospermum scoparium var. ‘triketone’.

In a further embodiment, the above tests may be completed in conjunction with other known region typing tests including but not limited to oxygen isotope analysis and trace element analysis. Such analyses may provide information on how far from the sea the honey has been collected from and what trace elements may have been available that are typical in the soil of the nectar producing plant. Such analyses may be particularly helpful if the honey is mainly from high yielding Leptospermum scoparium var. incanum that has been blended with a foreign honey which contains no phenolics or MGO. Such a honey blend could be detected by the above methods.

The above methods are unexpected over the art as the art does not find a true correlation between honey phenolic markers and that observed in plant nectar and therefore places no reliance on such markers for determining honey origin (or other factors). The inventors found that there was indeed a strong correlation, particularly once the age of the honey was removed as a factor.

### Plant Origin

According to a sixth embodiment there is provided a method of determining the plant origin of a honey sample by measuring the concentration of free phenolic compounds in the honey and comparing the results to a control sample or samples.

According to a seventh embodiment there is provided a method of determining the plant origin of a honey sample by measuring the concentration of methylglyoxal in the honey and comparing the results to a control sample or samples.

As should be appreciated from the above description, it is possible to determine the plant origin of a honey. One application of this method is in quality control and determining that products labelled with high value honeys such as manuka honey are in fact sourced predominantly from manuka plants or other sources with high value.

In one embodiment, the concentration of phenyllactic acid and the sum of the principal phenolic components are used to determine and distinguish whether a honey is sourced from manuka or kanuka plants.

Preferably, in the above embodiment, the marker compound is 4-methoxyphenyllactic acid. The inventors have found that the concentration of 4-methoxyphenyllactic acid is consistently less than 1% of sum of principal phenolic compounds in a manuka honey but always around 10% or greater in a kanuka honey. As a result it is very easy to distinguish between these species.

As noted above, it is also possible to use MGO content as a marker of plant origin. The inventors have determined a relationship between the concentration of methylglyoxal and the sum of principal phenolic compounds in a naturally aged manuka honey. Were manuka and kanuka honeys compared, MGO is not present at all in kanuka honey provided a simple distinction between the two types of honey.

In a further embodiment, clover (Trifolium spp.) and rewarewa (Knightia excelsa) honeys do not contain elevated levels of phenolic compounds making these honeys distinguishable by the absence of such compounds.

In a further embodiment, kamahi (Weinmannia spp,) (Broom, 5. J.; Wilkins, A, L.; Lu, Y.; Edo, R. M. 1994. Novel nor-sesquiterpenoids in New Zealand honeys. The relative and absolute stereochemistry of kamahines; an extension of the Mosher method to hemiacetals. Journal of Organic Chemistry 59: 425-6430) and heather (Erica spp.) (Hyink, W. 1998. A chemical investigation of some New Zealand honeys, MSc Thesis. University of Waikato, Hamilton, New Zealand) honeys contain unique kamahines and ericinic acid respectively making these honeys easily distinguished.

Furthermore, the above examples where New Zealand honeys are distinguished should not be seen as limiting as the same principles can be used to distinguish between honey plant origins in other countries. For example, the phenolicompound profile for Australian Jellybush honey harvested from Leptospermum polygalifolium and Eucalyptus spp. honeys have been determined (Yao, L.; Datta, N.; Tomas-Barberan, F. A,; Ferreres, F.; Martos, I., Singannusong, R. 2003. Flavonoids, phenolic acids and abscisic acid in Australian and New Zealand Leptospermum honeys. Food Chemistry 81:159-168). These plant species exhibit significantly different phenolic profiles from New Zealand honeys and therefore differentiation will be possible for plant origin.

Again, the above methods are unexpected over the art as the art does not find a true correlation between honey phenolic markers and that observed in plant nectar and therefore places no reliance on such markers for determining honey origin (or other factors). The inventors found that there was indeed a strong correlation, particularly once the age of the honey was removed as a factor.

### Quality Control

According to an eighth embodiment, there is provided a method of determining whether or not a batch of honey has been manipulated and thereby rejecting or receiving the honey batch by the steps of;
(a) obtaining a sample or samples from the batch of honey of a known age;
(b) measuring the concentration of at least one phenolic compound in the honey sample or samples;
(c) determining whether or not the phenolic concentration agrees with a known linear correlation for the honey or honey blend with age and wherein;
   i. if the phenolic compound or compounds concentration is more than two standard deviations higher or lower than that predicted for the honey, the honey batch is rejected;
   ii. if the phenolic compound or compounds concentration is within two standard deviations of that predicted for the honey, the honey batch is accepted.

According to a ninth embodiment, there is provided a method of determining whether or not a batch of honey meets label declarations as to floral origin and regional origin by the steps of:
(a) obtaining a sample or samples from the batch of honey of a known age;
(b) measuring the concentration of at least one phenolic compound in the honey sample or samples;
(c) determining whether or not the phenolic compound or compounds concentration agrees with a known linear correlation for the honey or honey blend with age and wherein;
   i. if the phenolic concentration is more than two standard deviations higher or lower than that predicted for the honey, the honey batch is rejected as not being true to the label declarations;
   ii. if the phenolic concentration is within two standard deviations of that predicted for the honey, the honey batch is accepted as being true to the label declarations.

In the above embodiment, manuka derived honey may be distinguished from other honeys by measuring the concentration of 2-methoxybenzoic acid and comparing this to a known standard.

According to a tenth embodiment, there is provided a method of determining whether or not a batch of honey has been manipulated and thereby rejecting or receiving the honey batch by the steps of:
(a) obtaining a sample or samples from the batch of honey of a known age;
(b) measuring the concentration of methylglyoxal (MGO) and at least one phenolic compound in the honey sample or samples;
(c) determining whether or not the MGO concentration agrees with a known linear correlation for the honey or honey blend with age and phenolic concentration and wherein;
   i. if the MGO concentration is more than two standard deviations higher or lower than that predicted for the honey based on age and phenolic concentration, the honey batch is rejected;
   ii. if the MGO concentration is within two standard deviations of that predicted for the honey based on age and phenolic concentration, the honey batch is accepted.

As should be apparent from the above description, it is possible to use the methods of the present invention to make a number of quality control measures and decisions. This is important as the value of honey increases tremendously based on alleged medical efficacy and as a result, it is important to know that the value is indeed real rather than a manipulated or inferior honey.

### Optimisation

According to an eleventh embodiment, there is provided a method of optimising a blend of honeys to tailor and maximise medical potency of a honey blend by the steps of:
(a) sampling and identifying the phenolic concentration and MGO content of a selection of honeys;
(b) determining the desired medical potency of the honey blend from a selection of emphasising:
   i. anti-microbial effects;
   ii. immune stimulation effects;
   iii. anti-inflammatory effects; and
(c) mixing together honeys wherein;
   i. if an anti-microbial effect is to be emphasised, honeys with maximum MGO content are blended together;
   ii. if an immune stimulation effect is to be emphasised, honeys with intermediate concentrations of MGO and phenolic compounds are blended together;
   iii. if an anti-inflammatory effect is to be emphasised, honeys with maximum phenolic concentration are blended together.

In the above embodiment, the honey samples may also be analysed to determine the quantity of fungal derived complex carbohydrates in order to determine honeys that may be used to further emphasise an immune stimulation effect.

The inventor's have found that fungal material, for example yeasts, spores, fungal cellular compounds, in the environment may have a significant influence on the degree of immune stimulation caused by the honey, particularly when the honey is placed on a wound. Compounds have been identified by the inventor's in high immune stimulation honeys that are commonly associated with fungal cellular material. More specifically, the fungal cellular material may include complex carbohydrate compounds associated with the cell wall of fungal material. An unexpected result noted by the inventors was that not only were these fungal derived compounds present, but they also appeared to have a synergistic effect on immune stimulation. As may be appreciated, honey often contains LPS material in the form of cell wall debris, primarily from bacteria in the natural environment. LPS is known to have an immune stimulatory effect that is measurable and reproducible. Experiments undertaken by the inventor's identified that a similar immune stimulatory effect may be observed between LPS and the high fungal material containing honeys, yet the fungal material containing honeys required nearly zoo times less concentration than LPS to acquire the same stimulatory action as LPS. As may be appreciated, honey containing immune stimulation properties may be useful in at least wound dressing applications where the normal innate wound healing process needs to be stimulated in order to treat for example, a chronic wound.

As should be apparent, the above method may be used in the preparation and production of dressings to suit particular applications.

### Phenolic Compounds

In the above embodiments, the phenolic compounds may be in a form selected from the group consisting of: a free form, a complexed form and mixtures thereof.

Preferably, the phenolic compounds are selected from the group consisting of: phenolic acids, phenolic salts, phenolic esters, related polyphenolic compounds, and combinations thereof.

Preferably, the phenolic compounds are derived from tannin compounds. As noted above, a useful correlation is the comparison to wines where aging is associated with the development of flavour and aroma in red wines due to the release of phenolic groups from tannins.

Preferably, the phenolic compounds are methoxylated. As noted above, the prior art teaches some useful properties attributable to methoxylated compounds. The inventors have found that honey which includes methoxylated compounds exhibit useful medical and nutritional effects. By way of example, the inventors have analysed the phenolics prominent in manuka (Leptospermum spp.) and kanuka (Kunsea spp.) and a large number of these phenolics are methoxylated at one or more points of their phenol or acid group. Compounds such as gallic or benzoic acid are present mainly in their methoxylated form such as methoxybenzoic acid, methoxygallic acid, methyl syringate, methoxyphenylactic acid or syringic acid. Methoxylation is therefore a major feature of the phenolics that are prominent in the above species that are acknowledged to have a higher medical and nutritional activity. The inventor's findings in combination with the art mean that effects envisaged for medical and nutritional applications include:
Greater bioavailability due to the methoxylated compounds be able to enter the cell faster;
Longer bioavailability due to the methoxylated compounds having a much longer half life within cells to scavenge free radicals;
Phase II enzyme induction properties;

For honey wound dressing applications, the methoxylated compounds are also likely to have a much longer half life within wound exudate as they are not rapidly degraded.

Methoxylation also results in much longer lived molecules once they are in the cell.

Also unexpectedly, the inventors have found that methoxylated compounds are very well tolerated by the human cells (low toxicity) but not by bacterial and fungal cells that is highly advantageous in treating microbial infections.

In a further embodiment, methoxylated phenolics may represent greater than 10% wt of the total phenolic compound content in the composition. Preferably, this may be greater than 20% wt. Preferably, this may be greater than 30% wt.

In a further embodiment, honey produced from the method or plant contains at least 150 mg/kg of methoxylated phenolic compounds.

Examples of principal phenolic compounds may be selected from the group consisting of: phenyllactic acid, methoxylated phenyllactic acid, methoxylated benzoic acids, syringic acid, methyl syringate, isomeric forms of methyl syringate, and combinations thereof.

In one embodiment the free phenolic content may be measured indirectly by determining the sum of phenyllactic and 4-methoxyphenyllactic acids and derivatives thereof (particularly hydroxylated analogues). These may be increased in the plant nectar by 5-10,000 mg/kg. Examples of these compounds are illustrated below:

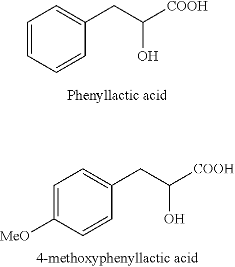

In a young honey these compounds are understood by the inventors to typically account for more than three-quarters of the principal phenolic components. The inventors have found that, with no other influences other than age, honey tend to show an increase in predominance of benzoic acid compounds and their derivatives.

Preferably, the methoxylated derivatives of benzoic acid noted above are: z-methoxybenzoic acid, 4-methoxybenzoic acid and isomers of trimethoxybenzoic acid as shown below:

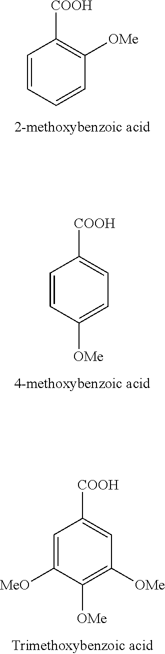

Hydroxylated benzoic acid derivatives (salicylic acid and 4-hydroxybenzoic acid) are also of interest although are present in less significant concentrations.

Preferably, the third group of the principal phenolic components noted above include syringic acid and methyl syringate:

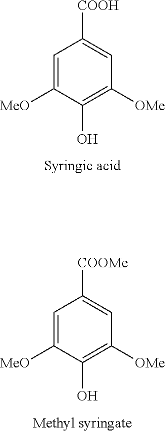

These components are present as two isomers that are diagnostic and differentiate manuka and kanuka honeys.

In a further embodiment, the free phenolics may also include a suite of other compounds allied with the tannin matrix in honeys. These range from relatively simple molecules such as gallic acid and methoxylated derivatives, abscisic acid, cinnamic acid, phenylacetic acid and methoxylated and hydroxylated derivatives, and methoxyacetophenone; to complexed polyphenolic molecules such as ellagic acid. A range of these molecules are illustrated below:

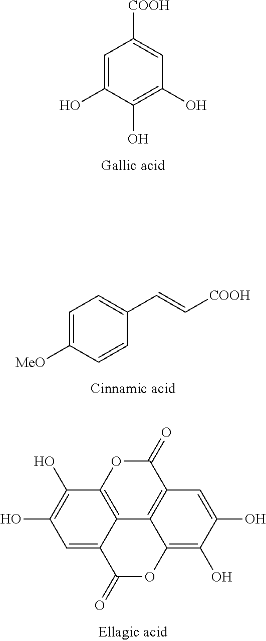

Preferably, the nectar contains free, complexed or a mix of phenolic compounds sufficient to results in honey with 5 mg/kg to 10,000 mg/kg or higher depending on the preferred application.

Preferably, the free phenolic content in the honey may be manipulated by addition of other components.

Probiotic bacteria or fungi may be useful in breaking down the tannin complex and increasing the number of free phenolic compounds in the honey. By way of example, Lactobacillus plantarum, a beneficial micro-organism that inhabits the human gut has been shown to degrade tannin complexes by catalysing the hydrolysis of ester and depside linkages in hydrolysable tannins into individual phenolic units thus freeing the biologically active units for cell absorption.

It should be appreciated from the above description that there are provided methods of analysing honey to determine various characteristics. These characteristics influence honey quality and the medical and/or nutritional potency of the honey. Advantages of such tests and manufacturing steps should be apparent including quality control tests that may be undertaken in the manufacture of honey and honey based compositions.

Embodiments of the method are now described with reference to various examples illustrating the medical and nutritional properties of the present invention.

### Example 3

In this example, honey harvested from the indigenous New Zealand shrubs Leptospermum scoparium (manuka) and Kunzea ericoides (kanuka) are used to demonstrate the presence of free phenolic compounds and the way the concentration of these compounds change over time. Manuka and kanuka honeys were chosen to illustrate this effect as they contain relatively high levels of free phenolics and derivative compounds compared to other honey types.

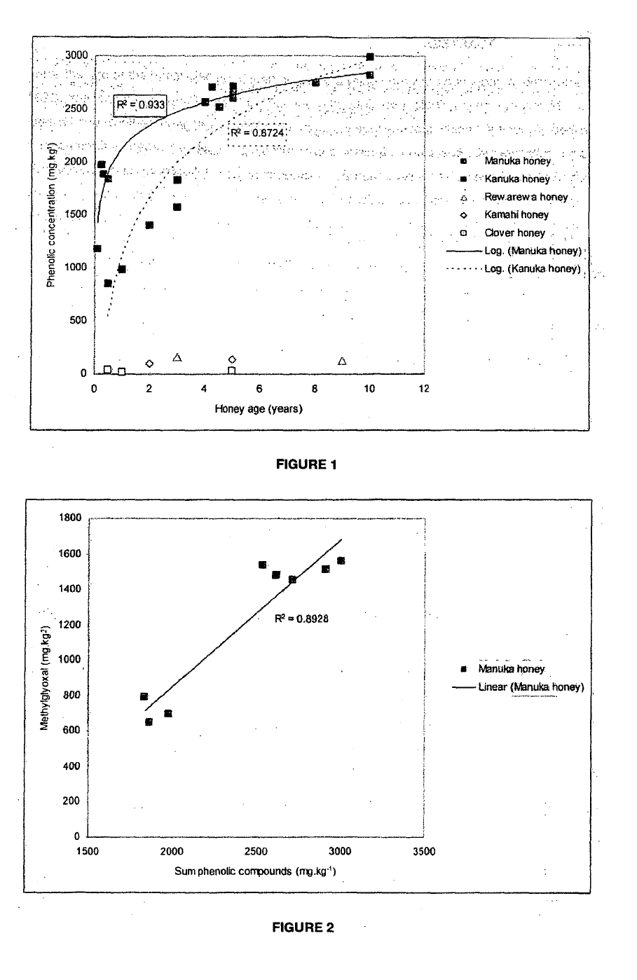

FIG. 1 a illustrates the concentration of the free phenolics present in five honey types of different ages. Relatively fresh (<3 months) manuka and kanuka honeys contain approximately 1000 mg. kg⁻¹ of these compounds, whereas in comparison the other honey types of the same age contain considerably less is than 100 mg. kg⁻¹. Furthermore as the manuka and kanuka honeys are aged naturally, that is stored at room temperature following extraction from the honey comb, the concentration of the phenolic components increases approximately three-fold over ten years to in the region of 3000 mg. kg⁻¹. However, the increase in free phenolic components' concentration illustrates a logarithmic curve; consequently much of the development of the phenolic profile occurs in the first five years of honey storage and aging.

Table 1 below describes the concentrations of these components during the aging process. Whilst these compounds are common to manuka and kanuka honeys, the concentration of some components differ significantly in these honeys.

TABLE 1
The phenolic profile and concentration of principal components mg/kg in monofloral
manuka and kanuka honeys harvested in New Zealand and aged naturally for ten years.

| | | | | | | | | Sum | |
|---|---|---|---|---|---|---|---|---|---|
| | | | | 4-methoxy- | Methoxylated | | | principal | |
| Honey | Age | | Phenyllactic | phenyllactic | benzoic | Methyl | Syringic | phenolic | Methy- |
| Type | (yr) | n | Acid | acid | acid | syringate | acid | components | glyoxal |
| Manuka | 0.5 | 3 | 1743 ± 77.5 | 4.8 ± 0.3 | 31.3 ± 4.1 | 19.3 ± 2.4 | 94.2 ± 8.0 | 1893 ± 78 | 714 ± 72.3 |
| | 5 | 3 | 1880 ± 40.0 | 4.9 ± 2.5 | 31.7 ± 3.4 | 310.7 ± 58.5 | 394.8 ± 32.4 | 2622 ± 91 | 1492 ± 45.0 |
| | 10 | 2 | 2001 ± 58.0 | 15.0 ± 4.2 | 33.5 ± 6.4 | 383.5 ± 40.3 | 520 ± 82.0 | 2953 ± 62 | 1538 ± 31.8 |
| Kanuka | 0.5 | 2 | 700.7 ± 26.1 | 93.3 ± 15.5 | 2.3 ± 0.8 | 63.3 ± 8.5 | 103.7 ± 11.9 | 963 ± 20 | 42.4 ± 23.4 |
| | 5 | 2 | 1549 ± 83.4 | 307.0 ± 21.2 | 3.4 ± 1.1 | 336.0 ± 12.7 | 592.5 ± 14.8 | 2788 ± 10.6 | 35.5 ± 26.2 |
| | 10 | 1 | 1680 | 512 | 7.2 | 338 | 554 | 3091 | 17.0 |

Values shown, mean ± standard deviation

The concentration of methylglyoxal in the manuka and kanuka honeys is also listed in Tablet Manuka honey, derived from Leptospermum scoparium, contains methylglyoxal. As a manuka honey is aged, the concentration of free methylglyoxal also increases in the honey. This increase is understood to be due to a different mechanism to the increase in phenolics owing at least to the way the compounds develop when heated. It is understood by the inventors that the MGO increase may be due to conversion of DHA to MGO.

FIG. 2 illustrates the correlation between the concentration of methylglyoxal and the principal phenolic compounds in a naturally aged manuka honey. Methylglyoxal and total phenolic compounds do not correlate in kanuka honey because the methylglyoxal component is derived from Leptospermum scoparium, and the small amounts of methylglyoxal in the kanuka honeys represent insignificant manuka honey contamination.

### Example 2

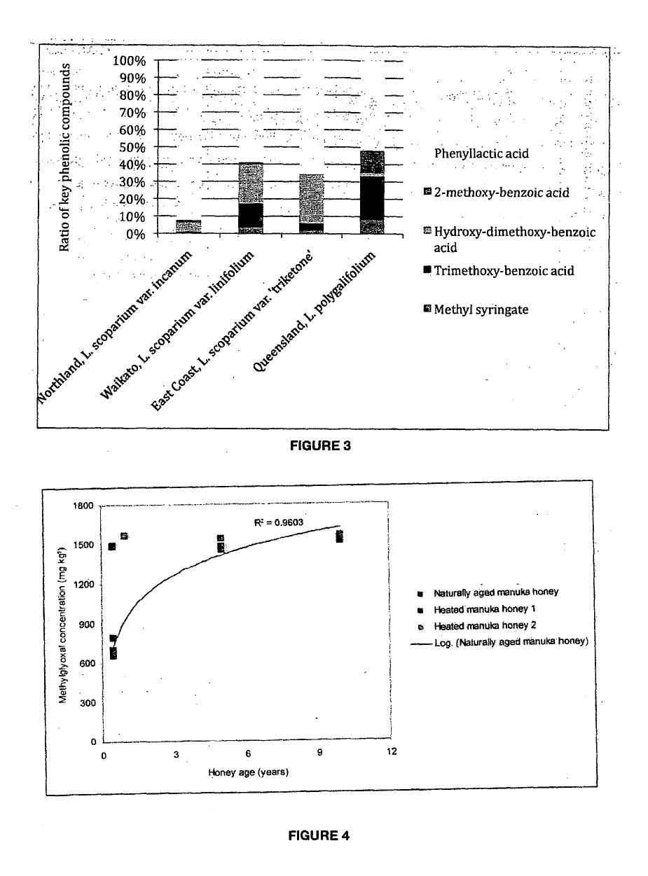

A further illustration of the presence of unique phenolic compounds in plant nectar used for honey manufacture is illustrated in FIG. 3 which shows a comparison between manuka honey produced from Northland, Waikato and East Coast in New Zealand and a sample from Queensland, Australia.

As can be seen in FIG. 3, the ratio of phenolic compounds allows separation by region, and botanic source. The concentration of 2-methoxy-benzoic and tri-methoxy-benzoic acids is significantly elevated in honey derived from Leptospermum polygalifolium in Queensland, Australia. Phenyllactic acid is elevated in honey from Northland, New Zealand where variety is Leptospermum scoparium var. incanum. Elevated tri-methoxy-benzoic acid separates honey sourced from the Waikato wetlands and the East Coast of the North Island, New Zealand.

### Example 3

In this example a range of honey samples were analysed to determine the antioxidant levels in the nectar derived honeys compared to control standards.

Antioxidant activity was determined by the ABTS assay using a spectrophotometric method for antioxidant activity using the ABTS radical assay (expressed as Trolox Equivalent Antioxidant Capacity) based on the method of Miller & Rice-Evans (Miller, N.J.; Rice-Evans, C.A. 1997: Factors influencing the antioxidant activity determined by the ABTS+radical cation assay. Free Radical Research 26(3): 195-199).

All samples were diluted with warm water as required to bring into the appropriate range for the assay.

The antioxidant activities of the various samples are given in Table 2.

TABLE 2
Antioxidant Levels for Honey Samples Tested

| | Antioxidant Activity | |
|---|---|---|
| | by ABTS Assay | |
| | (μmole TEAC/100 g) | |
| | | Standard |
| Sample Description | Average | Deviation |
| Far North, North Island NZ Honey (Fresh) | 131.4 | 3.7 |
| Far North, North Island NZ Honey (Aged) | 256.2 | 4.0 |
| Bush Blend Honey from Hokianga NZ (Fresh) | 176.4 | 9.2 |
| Bush Blend Honey from Hokianga NZ (Aged) | 189.9 | 0.8 |
| Waikato, NZ Wetlands Honey (Fresh) | 143.8 | 1.5 |
| Waikato, NZ Wetlands Honey (Aged) | 237.7 | 7.5 |
| East Coast, North Island NZ Honey (Fresh) | 153.9 | 4.3 |
| East Coast, North Island NZ Honey (Aged) | 243.7 | 3.4 |
| Kanuka Honey (Fresh) | 178.3 | 1.4 |
| Kanuka Honey (1 Year Old) | 148.5 | 7.4 |
| Kanuka Honey (2 Years Old) | 193.3 | 8.3 |
| Kanuka Honey (3 Years Old) | 239.6 | 11.4 |
| Heated Manuka Honey | 305.1 | 2.5 |
| Clover Honey | 49.7 | 3.2 |
| Rewarewa Honey | 215.9 | 3.4 |
| Standard - 2-methoxybenzoic, 80 mg/kg | 51.8 | 1.3 |
| Standard - phenyllactic acid, 210 mg/kg | 54.6 | 1.3 |
| Standard - methylsyringate, 290 mg/kg | 85.1 | 2.7 |
| Standard - gallic acid, 700 mg/kg | 1695.4 | 58.3 |
| Standard - syringic acid, 760 mg/kg | 499.6 | 25.3 |

As can be seen in Table 2, the antioxidant levels increase in honey with age supporting earlier Examples. This effect occurs irrespective of region from which the honey has been collected.

Also noted was that honeys known to have medical activity e.g. manuka honey, had moderate TEAC levels. Conversely, honeys known to have little medical activity e.g. rewarewa honey had higher TEAC counts. This variation in medical activity is understood by the inventors to be attributable to the phenolic levels (total TEAC count), but also the amount of methoxylated phenolic compounds. Manuka honey has been found by the inventors to have a high number of methoxylated phenolic compounds e.g. methoxybenzoic acid and methyl syringate. In contrast, honeys such as rewarewa have been found to contain fewer methoxylated phenolic compounds and more non-methoxylated phenolics such as gallic acid. As noted in the above description, methoxylated compounds appear to have a greater degree of potency.

### Example 4

In this example, tests were completed to confirm the presence of phenolic compounds in plant nectar from which honey is derived.

The phenolic components can be isolated from the nectar of plant varieties and species. Table 3 below illustrates some of the components isolated mg/kg from two distinct cultivars of Leptospermum scoparium, and Kunzea ericoides. All of the phenolic compounds that are present in the honeys are derived from these species and are present in the species' nectar.

TABLE 3
Phenolic components measured in cultivars of Leptospermum scoparium
and Kunzea encoides (mg/kg)

| | | Sum of | Sum of | | | |
|---|---|---|---|---|---|---|
| Plant | | Methyl | Syringic | 2-Methoxy- | 4-Methoxy- | |
| Variety/ | Phenyllactic | Syringate | Acid | benzoic | phenyllactic | Methyl- |
| Species | Acid | Isomers | Isomers | Acid | Acid | glyoxal |
| L. scoparium | 90 | 530 | 8.9 | 9.3 | — | 14 |
| cultivar 1 | | | | | | |
| L. scoparium | 450 | 330 | 8.6 | 11.8 | — | 32 |
| cultivar 2 | | | | | | |
| Kunzea ericoides | 380 | 850 | 14.3 | Trace | 72 | Nil |
| | | | | | | Detected |

Given that the honey bees perform about a ten-fold concentration of the nectar during the conversion into honey it is apparent three of these principal components are relatively more concentrated in the nectar than in the honey. This is evidence din vivo phenolic self-condensation reactions occurring as the honey bees perform nectar dehydration. Such in vivo self-condensation reactions have been well described in the study of aging in wine (Monagas, M,; Gomez-Cordoves C.; Bartolome, B. 2004. Evolution of the phenolic content of red wines from Vitis vinifera L. during ageing in bottle. Food Chem. 95(3) 405-412). In contrast syringic acid concentration is similar in nectar and fresh honey, indicating this molecule is mostly present as hydrolysable tannin in the nectar and the increased concentration in aged honey may be due to tannin body degradation.

The analysis of nectar components in various glasshouse conditions provides measurement of the plants production of the different components, and secondly production efficiency in different environments. This allows breeding selection to be tailored to fit the intended locations for plantation establishment.

### Example 5

As noted above in Example 3, methoxylated phenolic compounds appear to have a greater presence in honeys (and hence nectars from honeys) that are associated with greater medical activity e.g. manuka honey.

A further example is provided below demonstrating the quantity of methoxylated phenolic compounds in a variety of honeys and their comparative levels to further exemplify the presence of these methoxylated compounds in more ‘active’ honeys as opposed to less ‘active’ honeys.

In this example a wide range of honeys were tested using the same criteria to measure the presence and concentration of 2-methoxybenzoic acid as a representative methoxylated phenolic acid. The results found are shown below in Table 3.

TABLE 3
Honey and Methoxylated Phenolic Compound Concentrations

| | | Sample | | 2-Methoxy- |
|---|---|---|---|---|
| | | age | Geographic | Benzoic Acid |
| Honey | Principal floral origin (and possible floral contaminates) | (year) | origin | [mg/kg] |
| Manuka^{a} | L. scoparium var. incanum (Trifolium spp.) | 0.1 | Northland | 32.7 |
| Manuka^{a} | L. scoparium var. incanum (Trifolium spp.) | 0.5 | Northland | 28.9 |
| Manuka^{a} | L. scoparium var. incanum (Trifolium spp.) | 0.9 | Northland | 29.0 |
| Manuka^{b} | L. scoparium var. incanum (hive site not assessed) | 2.5 | Northland | 52.1 |
| Manuka^{b} | L. scoparium var. incanum (hive site not assessed) | 3.5 | Northland | 50.7 |
| Manuka^{b} | L. scoparium var. incanum (hive site not assessed) | 4.75 | Northland | 22.2 |
| Manuka^{b} | L. scoparium var. incanum (hive site not assessed) | 5 | Northland | 14.8 |
| Manuka^{b} | L. scoparium var. incanum (hive site not assessed) | 5 | Northland | 36.3 |
| Manuka^{a} | L. scoparium var. incanum (Trifolium spp., Knightia excelsa) | 0.4 | Northland | 5.7 |
| Manuka^{a} | L. scoparium var. incanum (Trifolium spp. K. excelsa, Kunzea | 0.75 | Northland | 4.3 |
| Manuka^{a} | L. scoparium var. linifolium (Trifolium spp., Weinmannia | 0.25 | Waikato | 22.2 |
| Manuka^{a} | L. scoparium var. linifolium (Trifolium spp., Weinmannia | 0.5 | Waikato | 23.3 |
| Manuka^{b} | L. scoparium var. linifolium (hive site not assessed) | 4 | Waikato | 4.5 |
| Manuka^{a} | L. scoparium var. myrtifolium (Trifolium spp., Knightia excelsa) | 0.5 | Whanganui | 1.2 |
| Manuka^{a} | L. scoparium var. triketone^{d} (Trifolium spp.) | 0.1 | East Coast | 5.9 |
| Manuka^{a} | L. scoparium var. triketone^{d} (Trifolium spp.) | 0.3 | East Coast | 6.4 |
| Manuka^{a} | L. scoparium var. triketone^{d} (Trifolium spp.) | 0.5 | East Coast | 6.4 |
| Manuka^{b} | L. scoparium var. triketone^{d} (hive site not assessed) | 5.5 | East Coast | 1.4 |
| Manuka^{b} | L. scoparium (variety unknown, hive site not assessed) | 1.5 | Unknown | 9.9 |
| Kanuka^{a} | Kunzea ericoides (Trifolium spp.) | 0.1 | Northland | Trace |
| Kanuka^{b} | Kunzea ericoides (hive site not assessed) | 1.5 | Northland | 0.7 |
| Kanuka^{b} | Kunzea ericoides (hive site not assessed) | 2.5 | Waikato | 0.3 |
| Kanuka^{b} | Kunzea ericoides (hive site not assessed) | 3.5 | East Coast | 1.1 |
| Clover^{c} | Trifolium spp. (hive site not assessed) | 1 | South Island | Trace |
| Rewarewa^{b} | Knightia excelsa (hive site not assessed) | 5 | Bay of Plenty | 0.4 |
| Nectar^{e} | L. scoparium var. incanum cultivar, 4 samples | — | Bay of Plenty | 17.7 (8.6) |
| Nectar^{e} | Leptospermum Nichollsii derived cultivar, 2 samples | — | Bay of Plenty | 7.6 (2.1) |
| Nectar^{ef} | Kunzea ericoides, 1 sample | — | Bay of Plenty | 0.5 |

^{a}Samples collected from hive sites;
^{b}Aged samples from drums supplied by apiarists and purchased as designated type;
^{c}Commercially labelled product;
^{d}Unclassified L. scoparium variety that carries an enhanced triketone essential oil profile;
^{e}Nectar samples collected from flowering specimen;
^{f}Qualitative measurement.

As shown in Table 3, the concentration of 2-methoxybenzoic acid is higher in manuka origin honeys than either kanuka, clover or rewarewa derived honeys suggesting methoxylated phenolic compounds may be important to medical efficacy.

### Example 6

In this example, tests to determine whether or not MGO has been added to honey or whether or not honey has been heated are illustrated.

Fortification of a manuka honey with methylglyoxal can be readily detected by the expected concentration of methyiglyoxal on the aging curve or a comparison between the expected concentrations of methylglyoxal and principal phenolic compounds in a manuka honey. The artificial addition of methylglyoxal to other honey types can also be detected.

The alteration of the profile by heating is similar to the artificial fortification with methylglyoxal; however heat treatment is readily detected by analysis of hydroxymethylfurfuraldehyde (HMF) value and a reduction in the honey enzyme invertase activity (Karabourniota, S.; Zervalaki, P. 2001. The effect of heating on honey HMF and invertase. Apiacta 36 (4), 177-181).

Furthermore it has been illustrated that heated honeys contain elevated levels, between two- and three-fold, of 3-deoxyglucosulose (3-DG) in association with hydroxymethylfurfuraldehyde, and more importantly these honeys do not develop methylglyoxal content despite being heated (Mavric, E. 2007. Argininderivatisierung and 1,2-D|carbonylverbindungen in Lebbensmitteln. PhD Thesis, Technische Universitat Dresden, Dresden, Germany), confirming the methylglyoxal content is derived from plant nectar rather than chemical reactions in the honey upon storage.

FIG. 4 illustrates the concentration of methylglyoxal in naturally aged manuka honeys harvested from Leptospermum scoparium. Two manuka honeys that are aged between 6 months and 1 year and were heated at approximately 30° C. for three months after extraction by the apiarist are also plotted, and these honeys significantly deviate from the standard curve.

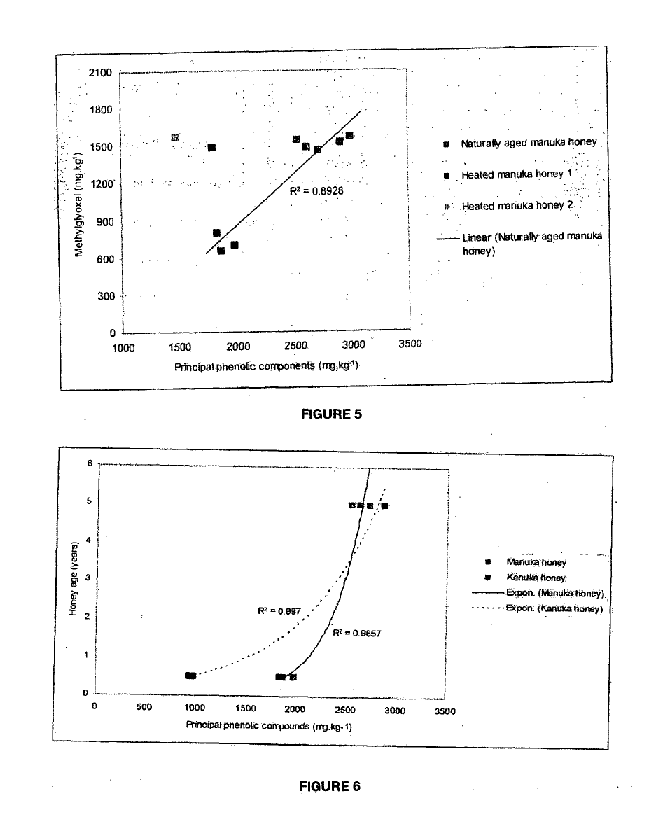

FIG. 5 illustrates the relationship between the concentration of principal phenolic compounds and methylglyoxal in naturally aged manuka honeys. The two honeys that have received an artificial heat treatment contain a significantly greater concentration of methylglyoxal.

As should be apparent from review of the above is that it is possible to determine whether a honey sample has been fortified with MGO or heat treated by reference to a known and untreated honey. This is because MGO and phenolic concentrations in the honey change in a predictable way overtime and as illustrated in the above graphs, variations to this natural process are obvious.

### Example 7

In this example, a test for regional variation is described.

As noted in the above description, there are also regional differences in the phenolic profiles of different types of honey.

In this example manuka honey harvested in New Zealand is referred to.

The inventors have analysed various manuka honeys where the variety of manuka plant from which the honey was derived were known. The separation of these Leptospermum scoparium varieties is in accordance with the divisions derived from essential oil chemotaxonomy and population genetics classification previously outlined (Stephens, J. M. C. 2006. The factors responsible for varying UMF levels in manuka (Leptospermum scoparium) honey, PhD Thesis. University of Waikato, Hamilton, New Zealand).

An illustrative finding is that Leptospermum scoparium var. incanum and Leptospermum scoparium var. linifollum exhibit similar profiles with a relatively more elevated ratio of methoxylated benzoic acids. By comparison, Leptospermum scoparium var. myrtifollum and Leptospermum scoparium var. ‘triketone’ have lower levels of methoxylated benzoic acids. Since these varieties of manuka grow in different regions in New Zealand it is therefore possible to identify regional characteristics in honeys based on their phenolic profiles.

### Example 8

In this example, age tests are illustrated along with some consideration as to the accuracy with which honey age may be determined.

As noted above, the age of a honey can be determined based on the finding that phenolic levels in honey change overtime in a measurable and consistent manner. Manuka or kanuka honeys are used below to illustrate this finding. Standard curves have been produced for these honeys by the inventors derived from the concentration of the principal phenolic components in the different honeys.

The data is transposed and accordingly the age becomes the predictive value, and equations establishing lines of best fit for the concentration of the principal phenolic compounds through the initial five years calculated. The results are shown in FIG. 6.

The results found above are now applied to an unknown honey sample.

As detailed in Table 3 below, the method is applied to two relatively monofloral manuka and kanuka honeys with an unknown age. An age is predicted based on the concentration of the principal phenolic compounds and then compared to the known age. The resolution using this method appears reasonable, as the calculated age values fell within the 95% confidence interval of accuracy.

TABLE 3
The application of the principal phenolic compounds standard
curves to predict the age of manuka and kanuka samples.

| Manuka honey, sample 1 | | Kanuka honey, sample 1 | |
|---|---|---|---|
| | Predicted | | Predicted |
| Principal phenolic | age | Principal phenolic | age |
| compounds (mg/kg) | (years) | compounds (mg/kg) | (years) |
| 2246 | 1.69 | 2418 | 3.46 |

It is expected that honey blends could also be tested using a similar process i.e. comparison to a known standard based on the same underlying principle of the phenolic levels changing over time. Blended honeys would require additional sets of standard curves to be prepared, and would employ a representative range of dilutions of manuka and kanuka honeys with the common forest and clover honeys harvested in New Zealand.

### Example 9

In this example, the method of determining the plant origin of a honey sample by measuring the content of phenolic compounds in the honey and comparing the results to a control sample or samples is illustrated.

The ratio of selected phenolic components, along with methylglyoxal, can be employed to determine the purity of honeys.

In this example, manuka and kanuka honeys are used to illustrate this effect.

The concentration of phenyllactic acid and the sum of the principal phenolic components correlate strongly for both manuka and kanuka honeys. As a result, either value can be applied to the calculations in determining plant origin. These ratios remain fairly constant throughout the aging process.

For example, the concentration of 4-methoxyphenyllactic acid is one of the most useful phenolic component indicators of purity. The concentration of 4-methoxyphenyllactic acid is consistently less than 1% of sum of principal phenolic compounds in a manuka honey but always around 10% or greater in a kanuka honey. As a result it is very easy to distinguish between these species.

Likewise the presence of methylglyoxal is a key indicator of the purity of a manuka honey. FIG. 2 illustrates the relationship between the concentration of methylglyoxal and the sum of principal phenolic compounds in a naturally aged manuka honey. The other floral honey types commonly harvested with manuka and kanuka honeys do not contain either the same phenolic compounds or methylglyoxal, and may also carry unique phenolic markers again helping to distinguish between honey plant origins. For example clover (Trifolium spp.) and rewarewa (Knightia excelsa) honeys do not contain elevated levels of the target phenolic compounds, and kamahi (Weinmannia spp.) (Broom, S. J.; Wilkins, A. L.; Lu, Y.; Ede, R. M. 1994. Novel nor-sesquiterpenoids in New Zealand honeys. The relative and absolute stereochemistry of kamahines: an extension of the Mosher method to hemiacetals, Journal of Organic Chemistry 59: 6425-6430) and heather (Erica spp.) (Hyink, W. 1998. A chemical investigation of some New Zealand honeys. MSc Thesis, University of Waikato, Hamilton, New Zealand) honeys contain unique kamahines and ericinic acid respectively making this easily distinguished.

Table 4 below lists the concentration of these components in four six-month old honeys; and provides a set of examples where the phenolic profile in association with the methylglyoxal concentration allows the prediction of the floral sources.

TABLE 4
The concentration of the principal phenolic* compounds (mg/kg)
in four six month old honeys harvested in New Zealand

| | | | | | | | 4-methoxy- | |
|---|---|---|---|---|---|---|---|---|
| | | | | | | Sum of | phenyllactic | |
| | | 4-methoxy- | Methoxylated | | | principal | acid as % of | |
| | Phenyllactic | phenyllactic | benzoic | Methyl | Syringic | phenolic | total phenolic | Methoxy- |
| Sample | acid | acid | acid | syringate | acid | compounds | compounds | glyoxal |
| 1 | 1720 | 6.5 | 25.2 | 28 | 104 | 1883.2 | 0.32 | 670 |
| 2 | 1380 | 54 | 13.8 | 38 | 106 | 1591.8 | 3.39 | 428 |
| 3 | 730 | 104 | 1.6 | 55 | 128 | 1018.6 | 10.21 | 14 |
| 4 | 380 | 48 | 0.6 | 24 | 58 | 510.6 | 9.40 | 0 |

*Principal phenolic compounds include phenyllactic acid, methoxylated phenyllactic acids, methoxylated benzoic acids, syringic acid, methylsyringate or its isomeric forms.

Sample a is a monofloral manuka honey as the ratio of 4-methoxyphenyllactic acid is less than 1% of total phenolic compounds, phenyllactic acid concentration is relatively high and methylglyoxal concentration fits the standard curve for manuka a honey.

Sample 2 is a manuka/kanuka blend honey; the 4-methoxyphenyllactic acid ratio falls between the monofloral manuka and kanuka predicted percentages, as does the concentration of phenyllactic acid, and the methylglyoxal concentration is approximately half of expected value.

Sample 3 is a monofloral kanuka honey; the 4-methoxyphenyllactic acid ratio is 1.0%, phenyllactic concentration acceptable and methylglyoxal is practically absent.

Sample 4 is a kanuka/clover blend honey; the concentration of the principal phenolic components is proportionally reduced, methylglyoxal is absent and the 4-methoxyphenyllactic acid ratio is almost 10%.

Clearly this method, with the development of a suitable database to act as a comparison allows for accurate determination of what plant species the honey in question is derived from.

Furthermore, the above example differentiating New Zealand honey plant origins should not be seen as limiting as the same principles can be used to distinguish between honey plant origins in other countries. For example, the phenolic compound profile for Australian Jellybush honey harvested from Leptospermum polygalifollum and Eucalyptus spp. Honeys have been determined (Yao, L.; Datta, N.; Tomas-Barberan, F. A. Ferreres, F.; Martos, I.; Singannusong, R. 2003. Flavonoids, phenolic acids and abscisic acid in Australian and New Zealand Leptospermum honeys. Food Chemistry 81: 159-168). These plant species exhibit significantly different phenolic profiles from New Zealand honeys and therefore differentiation will be possible for plant origin.

### Example 10

In this example the impact of heat on phenolic concentration and methylglyoxal is demonstrated.

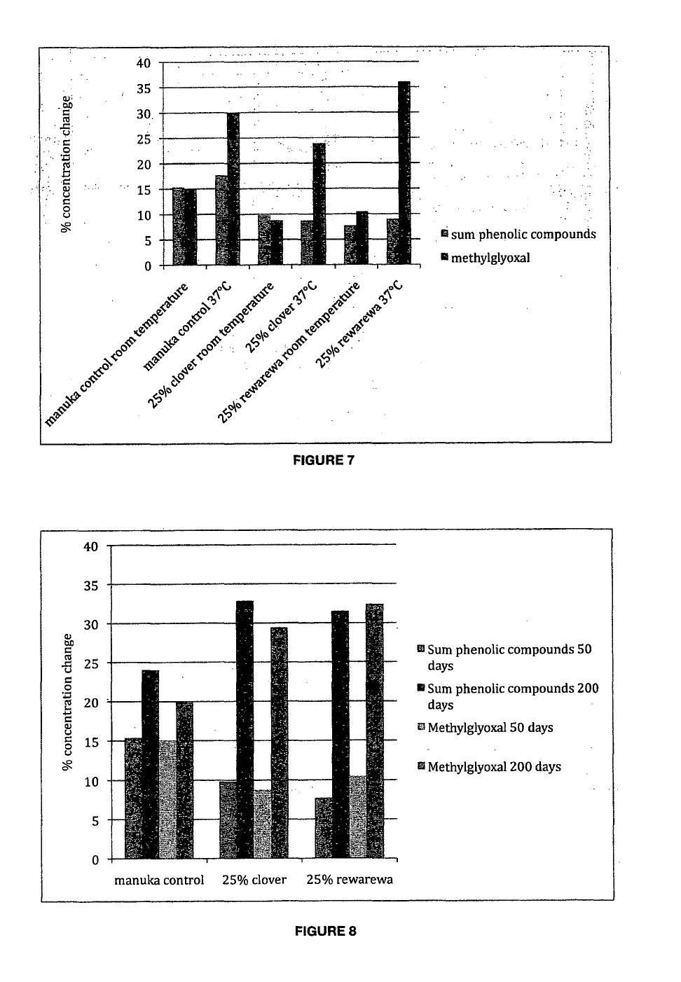

As shown in FIG. 7, the main effect noted was a significant change in MGO levels measured.

### Example 11

In this example, the rate of both methylglyoxal formation and available phenolic compounds and the influence of blend ratios and length of storage is shown, FIG. 8 illustrates the influence noted.

### Example 12

Acidification can be used to manipulate methylglyoxal concentration when honey stored at room temperature.

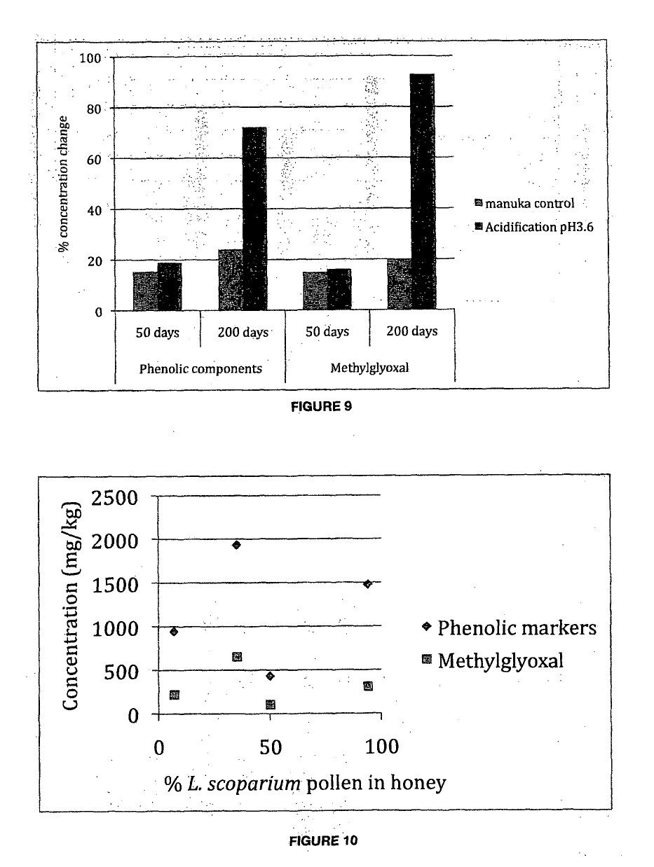

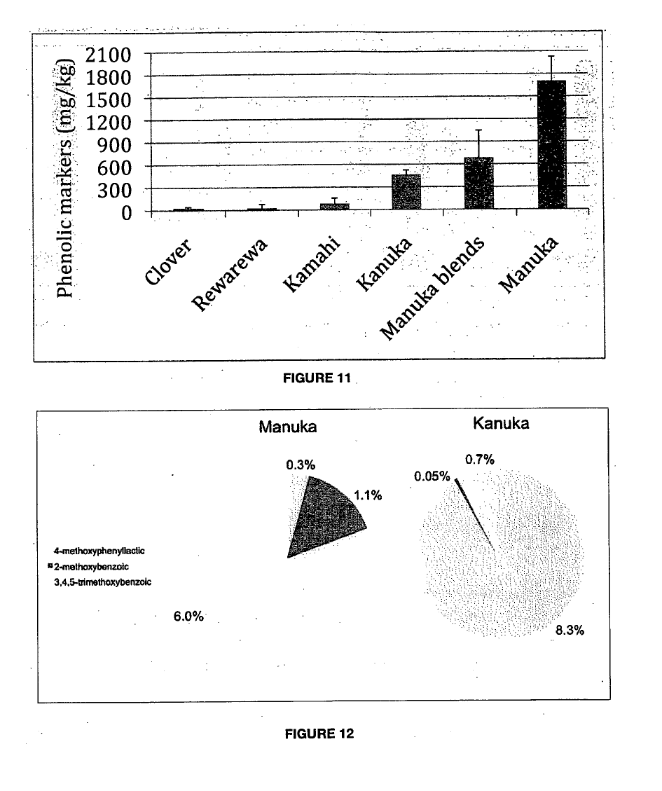

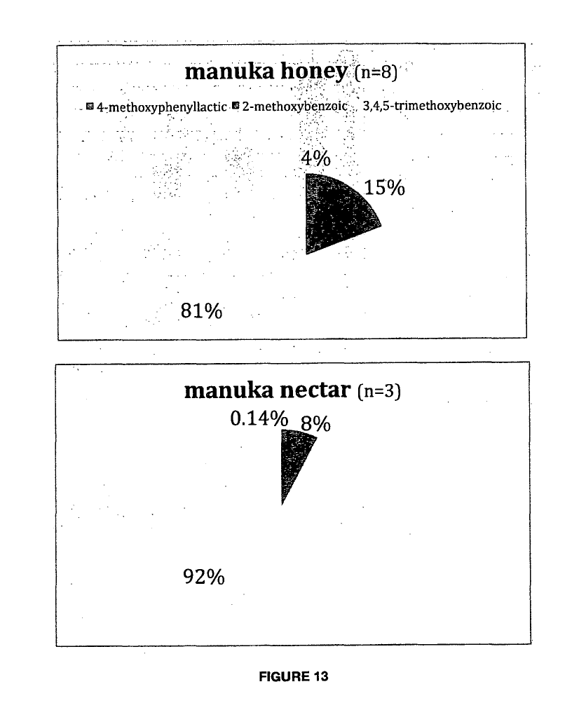

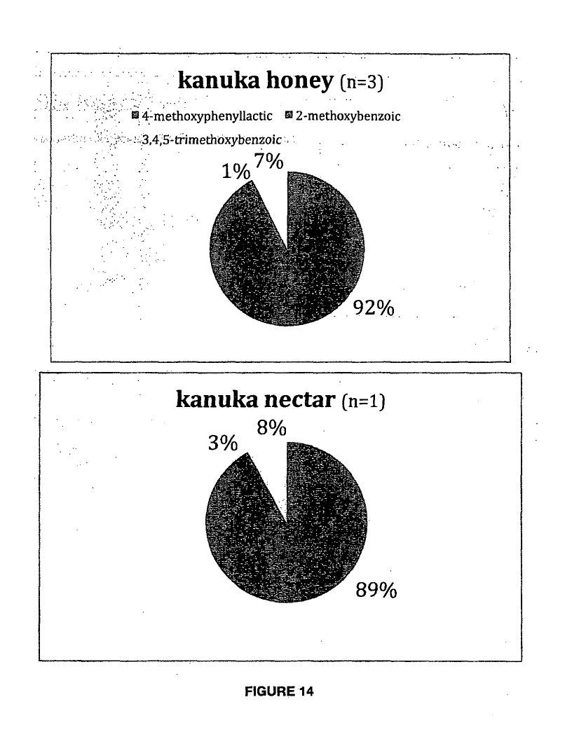

As shown in FIG. 9, acidification drammatically increased the concentration of both phenolic compounds and MGO in honey. Acidification was demonstrated to pH 3.6.

### Example 13

In this example a trial is demonstrated whereby a variety of bee pollen samples were collected and analysed to assess the concentration of a selection of key phenolic markers.

The key phenolic markers were phenyllactic acid, methoxyphenyllactic acid, 2-methoxybenzoic acid, 4-methoxybenzoic acid, syringic acid, methylsyringate, hydroxydimethoxybenzoic acid and trimethoxybenzoic acid.

The resulting concentrations were compared against that measured in honey of the same source to observe whether a correlation exists between the pollen and honey. MGO results were also taken as a further comparative measure.

As shown in FIG. 10, whilst the key phenolic markers were detected in both the pollen and honey, there is no correlation between the two with a wide spread of results. In addition, MGO results showed no correlation between the pollen and honey levels.

This example illustrates that pollen is not a reliable indicator of phenolic levels in honey unlike plant nectar demonstrated in earlier examples.

### Example 14

In this example a trial is demonstrated comparing the presence of different phenolic markers in various honeys.

Phenolic markers measured and illustrated are the same as those noted in Example 13 above.

The results found are illustrated in FIGS. 11 and 12.

As can be seen from the Figures, there are marked differences between different honey types. For example, manuka honey has a 2-3 fold greater concentration of phenolic marker compounds than kanuka honey. Also, manuka honey has a 3-fold greater concentration of 2-methoxybenzoic acid than kanuka honey.

Also, kanuka (and manuka) have markedly different concentrations of the key phenolic markers tested than other honeys including clover, rewa rewa and kamahi.

The above findings further demonstrate that it is possible to distinguish honey produced from different plant species. By way of example, manuka honey and the purity of the manuka honey may be determined by analysing the concentration of key phenolic marker compounds and/or by measuring the amount of 2-methoxybenzoic acid in the honey and comparing the results to a known database.

### Example 15

This example demonstrates further the correlation between key phenolic markers in nectar and honey.

As noted above, pollen phenolic concentration does not correlate well with honey phenolic concentration. In contrast, and as demonstrated above as well, a good correlation is observed between nectar phenolic concentration and honey phenolic concentration. This examples further illustrates this correlation by comparing samples of manuka honey and nectar as well as samples of kanuka honey and nectar.

As shown in FIG. 13 and FIG. 14, the comparative concentrations of three phenolic compounds were highly correlated in the honey and nectar in both manuka and kanuka thereby further illustrating the correlation between these two forms.

### Example 16

In this example, further details are provided as found by the inventor's for various properties of honeys. A wide variety of honeys were tested by the inventors as outlined already outlined in Example 5 above.

Further results found by the inventors for a variety of phenolic compounds and MGO are illustrated below in Table 5.

TABLE 5
Phenolic compounds and MGO present in various honeys

| | 3- | | 4- | |
|---|---|---|---|---|
| | methoxyphenyl- | Methyl | methoxyphenyl- | |
| Sample | lactic acid | Syringate | lactic acid | MGO |
| 1 | 5.2 | 5.1 | 5.8 | 658 |
| 2 | 91 | 5.4 | 5 | 651 |
| 3 | 103 | 6.3 | 4.5 | 793 |
| 4 | 8.1 | 11.2 | 2.8 | 1420 |
| 5 | 8 | 30 | 3.4 | 1080 |
| 6 | 429 | 168 | 5.6 | 1453 |
| 7 | 371 | 103 | 2.1 | 1541 |
| 8 | 9 | 50 | 36.3 | 425 |
| 9 | 97 | 15 | 8.1 | 218 |
| 10 | 320 | 99 | 25.2 | 297 |
| 11 | 357 | 56 | 9.6 | 512 |
| 12 | 369 | 74 | 9.2 | 783 |
| 13 | 334 | 111 | 25.6 | 1004 |
| 14 | 56 | 9.4 | 74 | 102 |
| 15 | 411 | 28 | 2.2 | 309 |
| 16 | 14 | 18 | 6.8 | 372 |
| 17 | 428 | 34 | 2.3 | 469 |
| 18 | 502 | 207 | 182 | 270 |
| 19 | 440 | 103 | 8.8 | 1490 |
| 20 | 7.8 | 39 | 13.9 | tr |
| 21 | 108 | 60 | 87 | 37 |
| 22 | 213 | 12 | 161 | 6 |
| 23 | 351 | 130 | 157 | 174 |
| 24 | 8 | 1.7 | 5 | nd |
| 25 | 4.6 | 12.7 | tr | nd |
| 26 | 4.3 | 11 | 0.6 | tr |
| 27 | 7.3 | 10.5 | tr | tr |
| 28 | 1 | 474 | 13.3 | tr |

As may be seen from the above results, a linear correlation is obvserved for honey between age and phenolic compounds such as 3-methoxyphenyllactIc acid, 4-methoxyphenyllactic acid, methyl syringate, and 2-methoxybenzoic acid. MGO also shows a linear correllation (when present) with an increase in age.

The above results also show how one variety may be distinguished from another. For example, manuka honeys tested had very different levels of 4-methoxyphenyllactic acid than kanuka honeys hence this phenolic is a candidate compound to identify when establishing the floral origin of a honey.

Aspects of the method described herein have been described by way of example only and it should be appreciated that modifications and additions may be made thereto without departing from the scope of the claims herein.

What is claimed is:

1. A method of determining whether or not a batch of honey has been manipulated and thereby rejecting or receiving the honey batch by the steps of:
(a) obtaining a sample or samples from the batch of honey of a known age;
(b) measuring the concentration of at least one phenolic compound in the honey sample or samples;
(c) determining, whether or not the phenolic concentration agrees with a known linear correlation for the honey or honey blend with age and wherein;
i. if the phenolic compound or compounds concentration is more than two standard deviations higher or lower than that predicted for the honey, the honey batch is rejected;
ii. if the phenolic compound or compounds concentration is within two standard deviations of that predicted for the honey, the honey batch is accepted.

2. A method of determining whether or not a batch of honey meets label declarations as to floral origin and regional origin by the steps of:
(a) obtaining a sample or samples from the batch of honey of a known age;
(b) measuring the concentration of at least one phenolic compound in the honey sample or samples;
(c) determining whether or not the phenolic compound or compounds concentration agrees with a known linear correlation for the honey or honey blend with age and wherein;
i. if the phenolic concentration is more than two standard deviations higher or lower than that predicted for the honey, the honey batch is rejected as not being true to the label declarations;
ii. if the phenolic concentration is within two standard deviations of that predicted for the honey, the honey batch is accepted as being true to the label declarations.

3. The method as claimed in claim 2 wherein manuka derived honey is distinguished from other honeys by measuring the concentration of 2-methoxybenzoic acid and comparing this to a known standard.

4. A method of optimising a blend of honeys to tailor and maximise medical potency of a honey blend by the steps of:
(a) sampling and identifying the phenolic concentration and MGO content of a selection of honeys;
(b) determining the desired medical potency of the honey blend from a selection of, emphasising:
i. anti-microbial effects;
II. immune stimulation effects;
iii. anti-inflammatory effects; and
(c) mixing together honeys wherein:
i. if an anti-microbial effect is to be emphasised, honeys with maximum MGO content are blended together;
ii. if an immune stimulation effect is to be emphasised, honeys with intermediate concentrations of MGO and phenolic compounds are blended together;
iii. if an anti-inflammatory effect is to be emphasised, honeys with maximum phenolic concentration are blended together.

5. The method as claimed in claim 4 wherein the honey samples are also analysed to determine the quantity of fungal derived complex carbohydrates in order to determine honeys that may be used to further emphasise an immune stimulation effect.

6. A method of determining whether or not a batch of honey has been manipulated and thereby rejecting or receiving the honey batch by the steps of:
(a) obtaining a sample or samples from the batch of honey of a known age;
(b) measuring the concentration of methylglyoxal (MGO) and at least one phenolic compound in the honey sample or samples;
(c) determining whether or not the MGO concentration agrees with a known linear correlation for the honey or honey blend with age and phenolic concentration and wherein;
i. if the MGO concentration is more than two standard deviations higher or lower than that predicted for the honey based on age and phenolic concentration, the honey batch is rejected;
ii. if the MGO concentration is within two standard deviations of that predicted for the honey based on age and phenolic concentration, the honey batch is accepted.

7. The method, as claimed in any one of the above claims wherein the phenolic compounds are selected from the group consisting of: phenolic acids, phenolic salts, phenolic esters, related polyphenolic compounds, and combinations thereof.

8. The method as claimed in any one of the above claims wherein the phenolic compounds are derived from tannin compounds.

9. The method as claimed in claim 8 wherein the tannins are hydrolysable tannin compounds.

10. The method as claimed in any one of the above claims wherein the phenolic compounds are methoxylated.

11. The method as claimed in any one of the above claims wherein the phenolic compounds are selected from the group consisting of phenyllactic acid, methoxylated phenyllactic acid, methozylated benzoic acids, syringic acid, methyl syringate, isomeric forms of methyl syringate, and combinations thereof.

12. The method as claimed in claim 11wherein the methoxylated benzoic acids are 2-methoxybenzoic acid and 4-methoxybenzoic acid

13. The method as claimed in any one of the above claims wherein the phenolic compounds are also selected from the group consisting of: gallic acid and methoxylated derivatives, abscisic acid, cinnamic acid, phenylacetic acid, methoxylated and hydroxylated derivatives of phenylacetic acid, methoxyacetophenone, ellagic acid, and combinations thereof.

14. A method of determining the age of a honey sample by measuring the concentration of phenolic compounds in the honey and comparing this concentration to a honey with a known age.

15. A method of determining:
(a) whether a honey has been fortified with MGO;
(b) whether a honey has been heated;
(c) whether a honey has been acidified;
by the steps of:
(i) knowing the approximate age of the honey and measuring the concentration of phenolic compounds and/or MGO in the honey; and,
(ii) comparing the measured concentration of phenolic compounds and/or MGO against a control honey with a known age.

16. A method of determining:
(a) the regional origin of a honey sample;
(b) whether a honey sample is a blend of honeys from different regions;
(c) the plant origin of a honey sample;
by the step of measuring the concentration of phenolic compounds in the honey and comparing the results to a control sample or samples.

17. The method as claimed in claim 16 wherein the MGO content of the sample is also measured and used in conjunction with the phenolic concentration.

18. The method as claimed in any one of claims 14 to 17 wherein the phenolic compounds in the honey derived from the plant nectar are in a form selected from the group consisting of: a free form, a complexed form, and mixtures thereof.

19. The method as claimed in any one of claims 14 to 18 wherein the phenolic compounds are selected from the group consisting of: phenolic acids, phenolic salts, phenolic esters, related polyphenolic compounds, and combinations thereof.

20. The method as claimed in any one of claims 14 to 19 wherein the phenolic compounds are derived from tannin compounds.

21. The method as claimed in claim 20 wherein the tannins are hydrolysable tannin compounds.

22. The method as claimed in any one of claims 14 to 20 wherein the phenolic compounds are methoxylated.

23. The method as claimed in any one of claims 14 to 21 wherein the phenolic compounds are selected from the group consisting of: phenyllactic acid, methoxylated phenyllactic acid, methoxylated benzoic acids, syringic acid, methyl syringate, isomeric forms of methyl syringate, and combinations thereof.

24. The method as claimed in claim 23 wherein the methoxylated derivatives of benzoic acid are benzoic acid; 2-methoxybenzoic acid and 4-methoxybenzoic acid

25. The method as claimed in claim 16 wherein manuka derived honey is distinguished from other honeys by measuring the concentration of 2-methoxybenzoic acid and comparing this to a known standard.

26. The method as claimed in any one of claims 14 to 25 wherein the phenolic compounds increased in the plant nectar also include phenolic compounds selected from the group consisting of: gallic acid and methoxylated derivatives, abscisic acid, cinnamic acid, phenylacetic acid, methoxylated and hydroxylated derivatives of phenylacetic acid, methoxyacetophenone, ellagic acid, and combinations thereof.
